(19) United States
(12) Patent Application Publication
(10) Pub. No.: US 2022/0061728 A1
(43) Pub. Date: Mar. 3, 2022

(54) INTEGRATED BRAIN MACHINE INTERFACE PLATFORM WITH GRAPHENE BASED ELECTRODES

(71) Applicant: Brain Scientific, Inc., Bohemia, NY (US)

(72) Inventor: Boris Goldstein, New York, NY (US)

(21) Appl. No.: 17/410,255

(22) Filed: Aug. 24, 2021

Related U.S. Application Data
(60) Provisional application No. 63/070,749, filed on Aug. 26, 2020.

(51) Int. Cl. A61B 5/291; A61B 5/263; A61B 5/00; A61B 5/282; A61B 5/296; A61B 5/251

(52) U.S. Cl. CPC A61B 5/291; A61B 5/263; A61B 5/0006; A61B 5/282; A61B 5/296; A61B 2562/125; A61B 5/4094; A61B 5/4064; A61B 5/746; A61B 5/251; A61B 5/4088

(57) ABSTRACT

This invention concerns a system for brain signal measurement and analysis using graphene based electrodes. The system is minimally invasive with small size electrodes and a stamp-size electronic processor with wireless communication and a remote computing device, enabling brain signal collection outside of clinical settings. The electrodes and electronic processor are both imprinted onto the subject's scalp using three-dimensional printers with small size electronics. After use, the electrodes and electronic processor may be washed off or removed without injuries to the subject.

### CROSS-REFERENCE TO RELATED APPLICATIONS

This application claims the benefit of U.S. Provisional Application No. 63/070,749, filed Aug. 26, 2020.

### BACKGROUND OF THE INVENTION

### Field of the Invention

This invention concerns a brain machine interface platform for brainwave measurement, where electrodes collect brain signals and transmit collected signals to a processing center attached to a human head. Brain signal collection and measurement have many applications, including studies on epilepsy, Alzheimer disease, stroke, and other brain disorders.

### Description of the Related Technology

Electroencephalography (EEG) is the recording of electrical signals along the scalp. Brains' neural activities generate electrical voltage fluctuations, whose signals may be measured by EEG. EEG measurements are useful for medical diagnosis and behavioral therapy. Other medical techniques involving the recording of bio-potential signals are electrocardiograms (ECG) and electromyograms (EMG).

Electroencephalography is particularly useful in diagnosis of conditions relating to brain injuries, such as seizure, stroke, brain tumors, Alzheimer's disease, or certain psychoses. Neural activities generate bio-potentials, which are collected by electrodes situated by a cap or by application of each electrode on certain head regions and conducted through electrical connections to a process hub.

EEG measurements typically require application of electrodes to the subject's head by either a cap application or placement of each electrode. Placement of each electrode is time consuming and requires a trained technician. Moreover, reusable caps and electrodes require cleaning and adding of gel, which may be time consuming and a means for germ transmission.

EEG measurements typically require the presence of the subject with healthcare provider(s) and required equipment, including the EEG machine, electrodes, and wirings. In many cases, EEG measurements are required when a specific event happens, such as a seizure, and measuring brain signals during these events prove to be challenging with the currently available technology and set up. Typically patients are required to stay in the hospital for observation and measurement when the opportunity arises.

Placement of electrodes for EEG measurement remains an obstacle. While the 10-20 international system for electrode placement has long been recognized, actual placement of electrode faces many challenges due to hair on the scalp. The need for gel to attach the electrodes further complicates the placement process. Connecting electrodes to the EEG machine has been by wires, which is another inconvenience.

There remains a need for a system and method to conduct EEG measurement using remote wireless connection with minimally invasive electrodes and small sensors, enabling the collection of EEG signals in any settings and at any time.

### SUMMARY

This invention provides a system and method for EEG measurement using small size electrodes and an EEG processor imprinted onto a subject's head. Electrodes are positioned and imprinted directly by three-dimensional printing onto the subject's scalp. Components of the EEG processor are also imprinted onto the subject's scalp and connected to the electrodes by imprinted connectors. The size of the EEG is about the size of a stamp. This system allows for continuous EEG measurement while the subject continues to maintain normal activities.

In particular, this invention provides a system for brain signal measurement, comprising:

a plurality of electrodes attached to the scalp of a subject, the plurality of electrodes comprising graphene and an epoxy material;

an electronic processor operatively connected to the plurality of electrodes, the electronic processor comprises:
at least one printed antenna configured to wirelessly communicate and transmit signals with outside electronic devices;
at least one printed battery configured to provide energy for operation of the electronic processor;
at least one sensor;
a central processor operatively connected to the at least one sensor and the at least one printed antenna to receive signals, process, and transmit received signals;
printed circuitry connecting the at least one printed antenna, the at least one printed battery, the at least one sensor, and the central processor; and
a connector;

printed circuitry connecting the plurality of electrodes and the electronic processor; and

at least one remote computing processor with an embedded computing programing product operatively connected to the electronic processor;

wherein the at least one printed battery comprises graphene material,

wherein the plurality of electrodes and electronic processor are imprinted onto the subject's scalp; and

wherein the electronic processor is configured to communicate with the at least one remote computing processor wirelessly.

This invention provides a system as above, wherein the plurality of electrodes are imprinted on the scalp by three-dimensional printing.

This invention provides a system as above, wherein the plurality of electrodes are implanted onto the skin on the subject's scalp.

This invention provides a system as above, wherein the plurality of electrodes are of the size between 5 μm-500 μm.

This invention provides a system as above, wherein the electronic processor is of the size between 1-2 centimeters in length and width, and 0.1-5 mm in thickness.

This invention provides a system as above, wherein the at least one sensor is at least one of electroencephalogram sensor, electrocardiogram sensor, or electromyography sensor.

This invention provides a system as above, wherein the remote computing processor is further configured to analyze data collected from the electronic processor.

This invention provides a system as above, wherein the remote computing processor further comprises a normative database.

This invention provides a system as above, wherein the normative database further comprises specific data encoding brain diseases.

This invention provides a system as above, wherein the diseases are epilepsy, Alzheimer disease, neurodegenerative disease, and stroke.

This invention provides a system as above, wherein the remote computing processor is further configured to aggregate data collected from the subject.

This invention provides a system as above, wherein the remote computing processor is further configured to analyze data collected from the subject and produce at least one output.

This invention provides a system as above, wherein the at least one output is an alert of an upcoming seizure episode.

This invention provides a system as above, further comprising a three-dimensional printers configured to print graphene electrodes on a subject's scalp.

This invention provides a method to collect brain signals, comprising:

providing a system comprising:
a plurality of electrodes attached to the scalp of a subject, the plurality of electrodes comprising graphene and an epoxy material;
an electronic processor operatively connected to the plurality of electrodes, the electronic processor comprises:
   at least one printed antenna configured to wirelessly communicate and transmit signals with outside electronic devices;
   at least one printed battery configured to provide energy for operation of the electronic processor;
   at least one sensor;
   a central processor operatively connected to the at least one sensor and the at least one printed antenna to receive signals, process, and transmit received signals; and
   printed circuitry connecting the at least one printed antenna, the at least one printed battery, the at least one sensor, and the central processor; and
   a connector;
printed circuitry connecting the plurality of electrodes and the electronic processor; and
at least one remote computing processor with an embedded computing programing product operatively connected to the electronic processor;
wherein the at least one battery comprises graphene material;
wherein the plurality of electrodes and electronic processor are imprinted onto the subject's scalp;
wherein the electronic processor is configured to communicate with the at least one remote computing processor wirelessly; and
a connector;

connecting the at least one remote computing device with the electronic processor using the embedded computing programming product;

collecting brain signals from the subject; and

recording the collected signals on the remote computing device as data.

This invention provides a method as above, further comprising the step of transmitting the data to another remote computing device and making the data available to authorized users.

This invention provides a method as above, wherein the authorized users are doctors, nurses, or researchers.

This invention provides a method as above, further comprising the step of analyzing the data to provide at least one output.

This invention provides a method to produce a brain measurement device, comprising:

providing a plurality of electrodes comprising graphene and an epoxy material;

providing an electronic processer, the electronic processor comprises:
at least one printed antenna configured to wirelessly communicate and transmit signals with outside electronic devices;
at least one printed battery configured to provide energy for operation of the electronic processor;
at least one sensor;
a central processor operatively connected to the at least one sensor and the at least one printed antenna to receive signals, process, and transmit received signals;
printed circuitry connecting the at least one printed antenna, the at least one printed battery, the at least one sensor, and the processor; and
a connector;

providing at least one remote computing processor with an embedded computing programing product operatively connected to the electronic processor;

determining locations for electrodes on a subject's head;

using the three-dimensional printer to print the plurality of electrodes onto the subject scalp's skin at the locations;

using the three-dimensional printer to print the electronic processor onto the subject's scalp skin; and

using the three-dimensional printer to print circuitry to connect the plurality of electrodes with the electronic processor.

### ABBREVIATION

3D: three dimensional

ECG: Electrocardiogram

EEG: Electroencephalography

EMG: Electromyograms

mm: millimeter

μm: micrometer

NFC: Near Field Communication

PANI: polyaniline

PEDOT:PSS: poly(3,4-ethylenedioxythiophene) polystyrene sulfonate

### BRIEF DESCRIPTION OF THE DRAWINGS

FIG. 1 is a schematic drawing of an EEG imprint system on a subject's head.

FIG. 2 illustrates the channels in an EEG 10/10 system.

FIG. 3 illustrates a head scan to identify electrode positions on a subject's head.

FIG. 4 illustrates a process to select reference points after electrode position identification.

FIG. 5 illustrates the process of three-dimensional (3D) scan of a subject's head with identified electrode positions.

FIG. 6 illustrates the 3D printing of electrodes and EEG imprint onto a subject's head.

FIG. 7 illustrates the top view of the 3D electrode printing process.

FIG. 8 is an exemplary embodiment of a 3D printer used for the printing process described herein.

FIG. 9 a schematic drawing of an EEG electronic processor imprint.

FIG. 10 illustrates an exemplary EEG electronic processor imprinted on the scalp of a subject.

### DETAILED DESCRIPTION OF CERTAIN INVENTIVE EMBODIMENTS

The detailed description set forth below in connection with the appended drawings is intended as a description of exemplary embodiments of the invention and is not intended to represent the only embodiments in which the invention may be practiced. The term “exemplary” used throughout this description means “serving as an example, instance, or illustration,” and should not necessarily be construed as preferred or advantageous over other exemplary embodiments. The detailed description includes specific details for the purpose of providing a thorough understanding of the exemplary embodiments of the invention. In some instances, some devices are shown in block diagram form.

The drawings presented herein are not to scale. Where dimensions are given, it is for illustrative purposes only and such dimensions shall in no way limit the invention disclosed herein. It is to be understood that different dimensions are contemplated for the system according to embodiments.

Reference to an element by the indefinite article “a” or “an” does not exclude the possibility that more than one of the elements are present, unless the context clearly requires that there is one and only one of the elements. The indefinite article “a” or “an” thus usually means “at least one.” Additionally, the words “a” and “an” when used in the present document in concert with the words “comprising” or “containing” denote “one or more”.

As used herein, the term “subject” refers to a mammal, preferably human subject.

As used herein, the term “cloud” refers to servers that are accessed over the Internet and the software and databases that run on those servers.

As used herein, the term “connector” refers to a coupling device that joins electrical terminations to create an electrical circuit.

As used herein, the term “normative database” refers to a database containing anatomic values of theoretically normal patients by averaging the measurements of a large number of patients. This measurement can be used as a baseline to track a patient's response to an intervention with pharmaceutical or other treatments.

Embodiments of this application relate to a system for collection of brain signals, comprising electrodes, an electronic processor and a remote computing device. The system has minimally invasive electrodes made from graphene materials and a stamp-size electronic processor imprinted on the subject's scalp and wirelessly connected to a remote computing article.

EEG measurement is the collection of brain signals using electrodes and electronic processors. The International Standard System for electrode placement on a human head is also known as the 10-20 system with 21 channels. This system is expanded to the 10/10 system with 81 channels. The amount of electrodes required in each type of measurement vary and EEG measurement systems are typically tailored to accommodate different types of measurements.

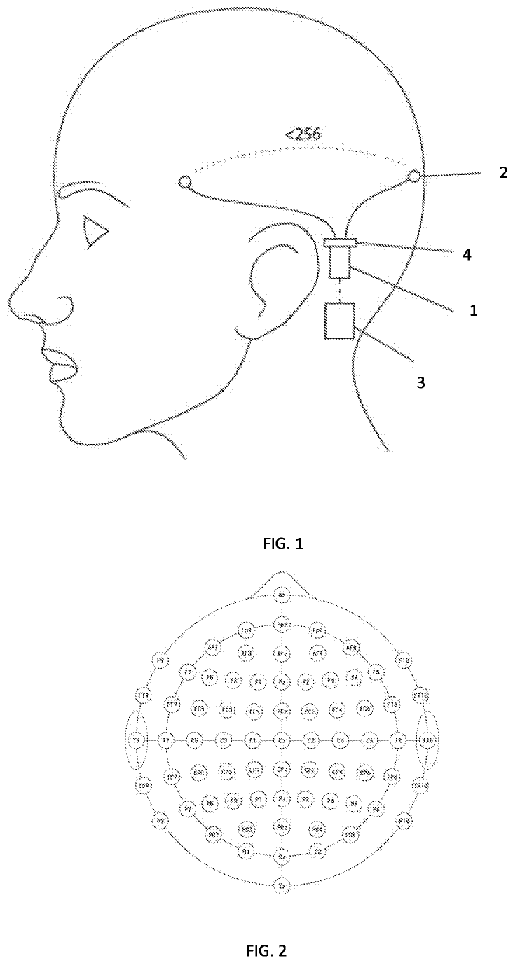

FIG. 1 illustrates the imprinted EEG system on a subject's head upon completion. The EEG electronic processor 1 may be of approximately stamp size with minimal thickness and may be imprinted by a 3D printer on the scalp near an ear. A connector 4 may be imprinted to be in contact with the central processor 8 and may be connected to electrodes 2 placed at various channels on the subject's head by imprinted connection, which may form imprinted circuitry. A cover 3 may be made available to cover up the EEG electronic processor 1 on the subject's scalp.

In embodiments, the system to collect brain signals may comprise electrodes 2 attached to desired locations on a subject's scalp. The electrodes 2 may comprise graphene material and is thinner than a human hair. In particular, the electrodes 2 may comprise a graphene-based matrix with high conductivity, stable mechanical structure and minimally irritable to the subject. Graphene may be cured in a matrix to increase mechanical stability while maintaining conductivity, flexibility, and stretching ability. Example of graphene materials are a combination of graphene or nanotubes, or any combination of graphene and/or nanotubes with other conductive additives, such as serpentine gold (Au) mesh, poly(3,4-ethylenedioxythiophene) polystyrene sulfonate (PEDOT:PSS), polyaniline (PANI)-based biodegradable composites, or silver/silver chloride (Ag/AgCl).

In embodiments, the electrodes 2 may be placed on the scalp at desired locations corresponding with the 10-20 or 10-10 system. In the first step, electrode locations may be determined for the particular purpose of the measurement. Some measurement types require more electrodes while others require less.

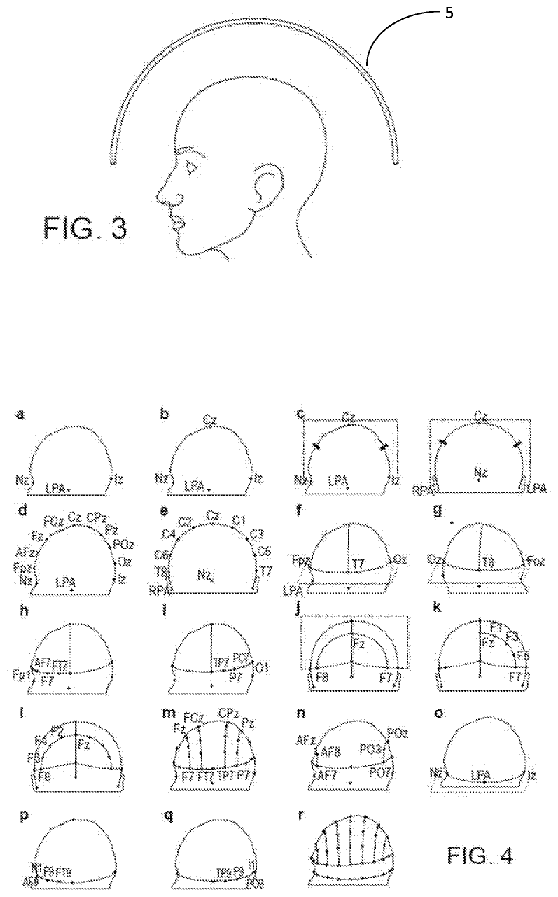

FIG. 2 illustrates the various channels on a subject's head in a 10/10 system. With this map, a subject's head may be scanned using an 3D scanner as in FIG. 3, such that a 10/10 system is identified on the subject's head to pinpoint electrode locations for each channel. FIG. 4 illustrates the results that may be collected from such a scan, where reference points are identified and selected on the subject's head upon scanning.

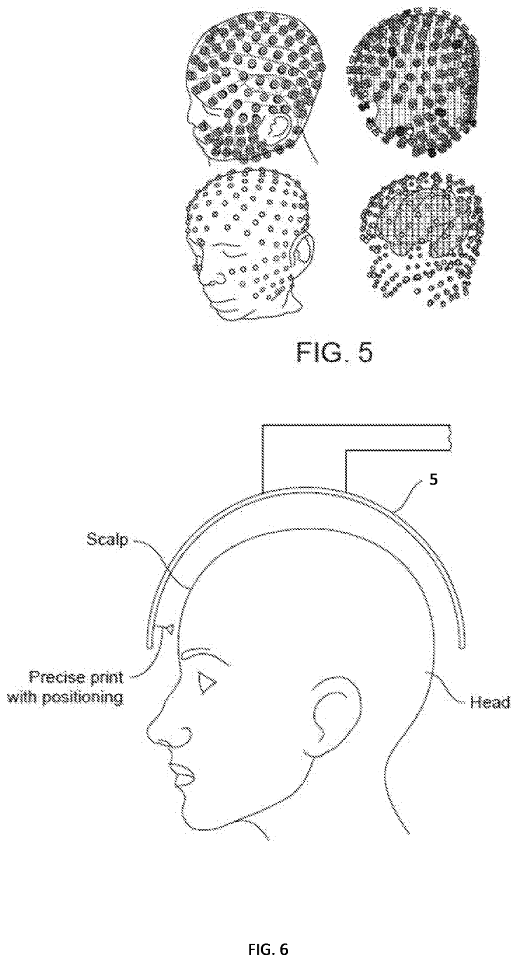

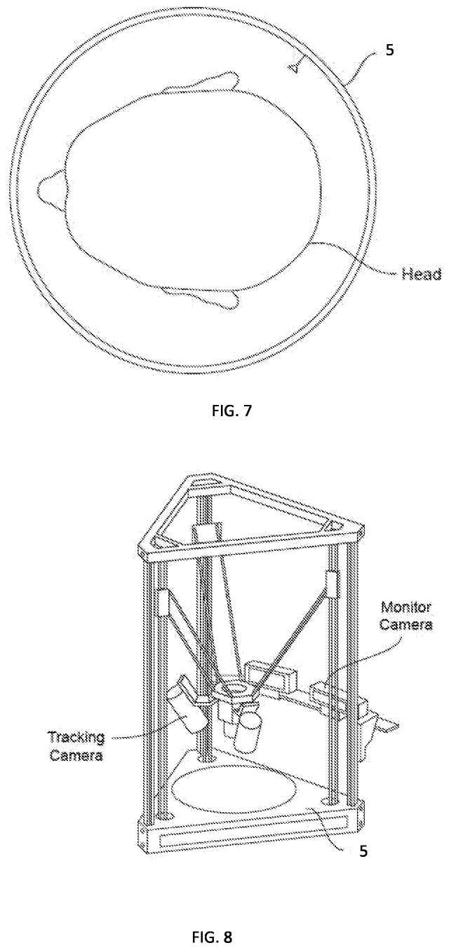

FIG. 5 illustrates exemplary 3D models of a subject's head with electrode positions identified using a combination of ultrasound, infrared 3D scan and/or structured light 3D scan. Various electrode positions in the 10/10 system are identified and personalized on the subject's head. This may allow precise position of electrodes by imprint.

Thereafter, the verification step may be carried out to ensure that the electrode locations identified correspond with the channels of the 10/10 or 10/20 system. Digitation reliability and validity may be verified in this step using a software connected to the 3D scanner to compare the channels according to the 10/10 system and those as identified by the 3D scanner. Upon successful verification, electrodes 2 may be placed on the subject's skin by imprinting onto the scalp.

FIG. 6 illustrates the process of electrode placement on a subject's scalp using a three-dimensional printer 5. The subject's hair may be parted at the locations where the electrodes 2 will be attached, and a three-dimensional printer 5 may be employed to print the electrodes at those locations. With the small size of the electrodes 2, precise placement is possible without the need for hair removal. The 3D printer 5 may be placed onto the subject's head in a similar manner to that of a hat with an electrode imprint mechanism inside. Alternatively, a simple 3D printer may be used, such that only the printing needle is used near the subject's head. Electrodes 2 printed onto the subject's scalp by a three-dimensional printer may be of a size between 5 μm (preferable for conformal imprint) up to 50-100 μm. If the electrode size is larger than 100 μm, connectivity to the skin may become non-conformal. However, the electrode size may still be up to 500 μm. Other dimensions are contemplated. In comparison, human hair varies in diameter, ranging anywhere from 17 μm to 181 μm. Electrodes 2 placed by 3D printers may be of substantially round shape rested on the skin. Other shapes may be formed depending on 3D printer, such as square, long rod, or cone, or other shapes, depending on the form of the scalp and the positioning of the electrodes 2. The shape of the electrodes 2 may need to conform to the contact point on the scalp, hence if the electrode position is at a place where the skull curves, the electrode contact point may need to conform to maintain contact with the scalp.

Electrodes 2 are printed by a 3D printer in a similar manner to a tattoo printed by a needle. In some embodiments, the electrodes 2 are not embedded under the dermis like a tattoo. In other embodiments, the electrodes 2 are embedded subcutaneously. For illustrative purposes, the following steps in imprinting an electrode 2 onto the skin are described. Materials to form the layers of electrode 2 are mixed in a nozzle before being applied to the scalp. Examples of such materials are serpentine gold (Au) mesh or poly(3,4-ethylenedioxythiophene) polystyrene sulfonate (PEDOT:PSS) or polyaniline (PANI)-based biodegradable composites, or silver/silver chloride (Ag/AgCl) or any combination of these materials with graphene and/or carbon nanotubes. This material mixture may be used as the “ink” to be printed by a 3D printer onto the subject's scalp to form the electrodes 2.

Alternatively, the electrodes 2 may be implanted under the skin on the subject's scalp. The electrodes 2 are thinner than a human hair and thus implantation under the subject's skin may result in minimal irritability. Graphene-epoxy materials are non-toxic and implantation may be carried out with minimal risk for the subject. For example, the size of the electrodes 2 are around 5 μm. Other dimensions are contemplated. These electrodes 2 are generally of rod-like shape. They may be implanted in a similar manner to that of acupuncture needles, where a thin needle-like electrode is inserted into the subject's scalp at each position. The thin rods may allow minimal discomfort to the subject.

FIG. 8 illustrates an exemplary 3D printer with a Delta robot to direct movement of the printing needle. The 3D printer may utilize a Delta parallel robot equipped with an injection needle to discharge an ink material for precise placement of the ink material at desired location. The material may form electrodes 2 and/or connectors upon being placed. Cameras may be present on the robot to track and monitor the progress of electrode and other component printing. An operator may view the progress on a screen with images fed by the cameras on the robot.

In embodiments, imprinted electrodes 2 and the electronic processor 1 may be connected by imprinted connection. The technique used may be similar to the technique to imprint electrodes 2. A layer of silicone may be imprinted onto the subject's skin, then a conductive ink layer, such as silicone/silver on top of the silicone layer, then another layer of silicone on top of the conductive layer to form imprinted connections in the nature of electronic circuitry. The imprinted connection may be printed by the 3D printer 5.

In embodiments, the system may further comprise an electronic processor 1. The electronic processor 1 may be the size of a stamp with similar thickness, even though other dimensions are contemplated to accommodate the components of the electronic processor 1. The size of the electronic processor 1 may give an advantage of feeling less cumbersome for the subject. This size may also allow the electronic processor 1 to be imprinted on the scalp next to the subject's ears and enable normal movement in everyday activities while wearing the electronic processor 1. In an exemplary embodiment, the size of the electronic processor 1 is between 1-2 centimeters in length and width and 0.1-5 millimeters in thickness.

The electronic processor 1 may comprise sensors 5 to collect data, a battery 7 to power the electronic processor's operation, an antenna 9, a central processor 8 to process and transmit collected data to a remote computing device, a connector 4 to connect the electronic processor 1 to the electrodes 2 imprinted on the scalp, and optionally a Near Field Communication (NFC) chip, among other components. These components may be separated into electrode layer, circuit layer, antenna layer, NFC charging layer, and processor layer. All components on the electronic processor may be imprinted onto the body of the electronic processor. Alternatively, all components of the electronic processor may be arranged and provided as one stamp-size equipment, which may be attached to the subject's scalp by an imprinted process. The electronic processor may also be implanted onto the subject's scalp.

The battery 7 and the sensors 10 may comprise graphene materials to reduce the size and improve conductivity. Sensors 10 may be EEG, ECG, EMG, or temperature sensors to collect various data from the subject, including brain signals and other physiological parameters. Other types of sensors may be used. The antenna 9, such as a Bluetooth antenna, on the electronic processor 1 may enable remote, wireless communication with another computing processor. Both the battery 7 and the sensors 10 may be printed on to the body using 3D printers.

The battery 7 may be graphene battery, which is small in size and suitable for imprint as part of the electronic processor. The battery 7 may be connected to the antenna 9 and the central processor 8 by imprinted circuitry to provide energy for the electronic processor's operation. Other small size batteries suitable for the purpose may also be used. Other components may be present to allow smooth operation of the electronic processor 1.

The electronic processor 1 may comprise a central processor 8 operatively connected to the sensors 10 and the antenna 9 and powered by the battery 7. Signals received by the electrodes 2 and/or sensors 10 may be transmitted by imprinted connections to the central processor 8, which may process the signals to transmittable data to be sent to a remote computing processor via the antenna 9. The central processor 8, sensors 10, antenna 9, and the battery 7 may be connected by printed circuitry among them, which may also be printed on the subject's scalp.

To imprint the electronic processor 1 onto the subject's scalp, a 3D printer 5 may be used. A layer of silicone may be printed onto the skin and small size components, such as the central processor 8, sensors 10, antenna 9, and the battery 7, may be placed onto the layer of silicone for attachment. Conductive ink may be placed using a small needle to inject ink onto the skin and connect the central processor 8 with other components. Imprinting of connections between the components in the electronic processor 1 may be similar to imprinting of connections from the electrodes 2. The battery 7, connector 4, and antenna 7 may be imprinted onto the subject's skin in similar manner.

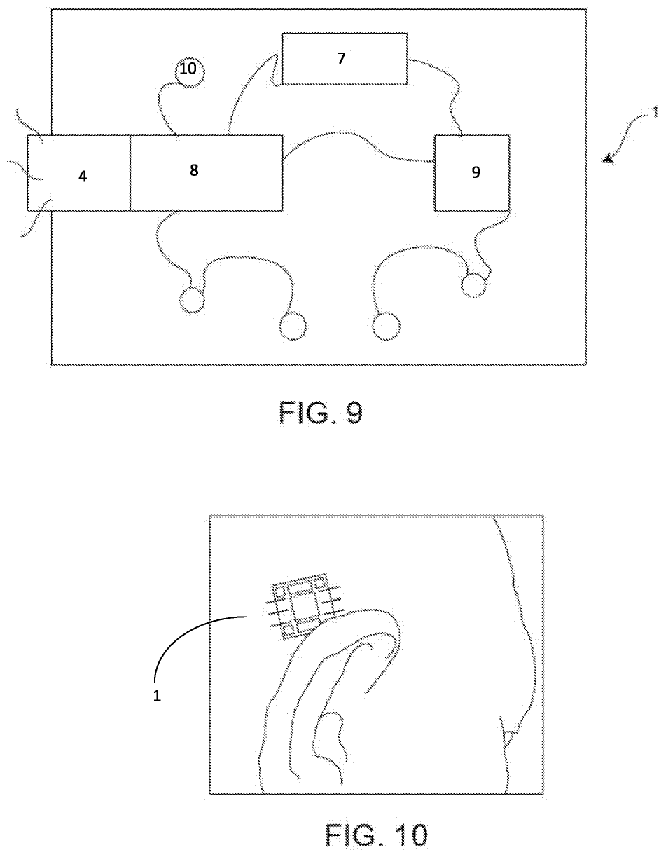

FIG. 9 is the schematic drawing of an imprinted electronic processor 1. The battery 7 may be connected to the central processor 8 and the antenna 9 using imprinted circuitry. The connector 4 may be in physical contact with the central processor 8 and may be connected to electrodes 2 by imprinted circuitry. The connector 4 may also be connected to the central processor 8 by printed circuitry or other means. The connector 4 may also operatively connect to the electronic processor 1 by other arrangements apart from being connected to the central processor 8. The sensors 10 may be placed at desired locations and connected to the antenna 9, the central processor 8, or each other using imprinted circuitry.

FIG. 10 illustrates an exemplary imprinted electronic processor 1 as present on a subject's scalp near the ear. The imprinted electronic processor 1 is of the size of a stamp with minimal thickness and is imprinted onto the scalp in a minimally invasive manner. The imprinted electronic processor 1 as a whole has the look of a tattoo but may be washed off after use.

The electronic processor 1 may be configured to communicate with a remote computing processor, which may be a desktop computer, a laptop, a smart phone, an iPad, or other computing devices, which may have an embedded computing programing product operatively connected to the electronic processor. Communication may be wirelessly through an antenna 9, such as a Bluetooth antenna, embedded in the electronic processor 1.

The remote computing processor may comprise a computer programming product in the nature of an application or a software configured to receive data from the electronic processor via the Bluetooth antenna. The computer programming product may be configured with a normative database to store information concerning normal operations of a healthy brain and data encoding brain diseases. The computer programming product may be further configured to analyze data received from the electronic processor and compare with the normative database to provide an output indicating the nature of data received. The computer programming product may indicate that the signals received correspond with epilepsy, Alzheimer disease, neurodegenerative disease, or stroke, among other diseases.

In embodiments, a normative database may be provided, which may contain data concerning normal operations of a healthy brain. The normative database may also contain data concerning various brain diseases. Comparison between data from the normative database and data collected from an particular subject may be used as basis for diagnosis, treatment, and study.

The computer programming product may be further configured to aggregate data collected from the subject and analyze to identify trend and prediction of future events, such as the likelihood of a future epilepsy episode. Data collected from one subject may also be shared via the Cloud with others, such as remote doctors, nurses, healthcare workers, or researchers for treatment decisions and research purposes. Data from multiple subjects may also be collected and aggregated to further study brain signals and related brain diseases.

All references, including publications, patent applications, and patents cited herein are hereby incorporated by reference to the same extent as if each reference were individually and specifically indicated to be incorporated by reference and were set forth in its entirety herein.

It will be readily apparent to those skilled in the art that a number of modifications and changes may be made without departing from the spirit and the scope of the present invention. It is to be understood that any ranges, ratios, and range of ratios that can be derived from any of the data disclosed herein represent further embodiments of the present disclosure and are included as part of the disclosure as though they were explicitly set forth. This includes ranges that can be formed that do or do not include a finite upper and/or lower boundary. Accordingly, a person of ordinary skill in the art will appreciate that such values are unambiguously derivative from the data presented herein.

What is claimed is:

1. A system for brain signal measurement, comprising:
a plurality of electrodes attached to the scalp of a subject, the plurality of electrodes comprising graphene and an epoxy material;
an electronic processor operatively connected to the plurality of electrodes, the electronic processor comprises:
at least one printed antenna configured to wirelessly communicate and transmit signals with outside electronic devices;
at least one printed battery configured to provide energy for operation of the electronic processor;
at least one sensor;
a central processor operatively connected to the at least one sensor and the at least one printed antenna to receive signals, process, and transmit received signals;
printed circuitry connecting the at least one printed antenna, the at least one printed battery, the at least one sensor, and the central processor; and
a connector;
printed circuitry connecting the plurality of electrodes and the electronic processor; and
at least one remote computing processor with an embedded computing programing product operatively connected to the electronic processor;
wherein the at least one printed battery comprises graphene material,
wherein the plurality of electrodes and electronic processor are imprinted onto the subject's scalp; and
wherein the electronic processor is configured to communicate with the at least one remote computing processor wirelessly.

2. The system of claim 1, wherein the plurality of electrodes are imprinted on the scalp by three-dimensional printing.

3. The system of claim 1, wherein the plurality of electrodes are implanted onto the skin on the subject's scalp.

4. The system of claim 1, wherein the plurality of electrodes are of the size between 5 μm-500 μm.

5. The system of claim 1, wherein the electronic processor is of the size between 1-2 centimeters in length and width, and 0.1-5 mm in thickness.

6. The system of claim 1, wherein the at least one sensor is at least one of electroencephalogram sensor, electrocardiogram sensor, or electromyography sensor.

7. The system of claim 1, wherein the remote computing processor is further configured to analyze data collected from the electronic processor.

8. The system of claim 7, wherein the remote computing processor further comprises a normative database.

9. The system of claim 8, wherein the normative database further comprises specific data encoding brain diseases.

10. The system of claim 9, wherein the diseases are epilepsy, Alzheimer disease, neurodegenerative disease, and stroke.

11. The system of claim 10, wherein the remote computing processor is further configured to aggregate data collected from the subject.

12. The system of claim 11, wherein the remote computing processor is further configured to analyze data collected from the subject and produce at least one output.

13. The system of claim 12, wherein the at least one output is an alert of an upcoming seizure episode.

14. The system of claim 1, further comprising a three-dimensional printer configured to print circuitry, graphene electrodes, sensors, antennas, and batteries on a subject's scalp.

15. A method to collect brain signals, comprising:
providing a system comprising:
a plurality of electrodes attached to the scalp of a subject, the plurality of electrodes comprising graphene and an epoxy material;
an electronic processor operatively connected to the plurality of electrodes, the electronic processor comprises:
at least one printed antenna configured to wirelessly communicate and transmit signals with outside electronic devices;
at least one printed battery configured to provide energy for operation of the electronic processor;
at least one sensor;
a central processor operatively connected to the at least one sensor and the at least one printed antenna to receive signals, process, and transmit received signals;
printed circuitry connecting the at least one printed antenna, the at least one printed battery, the at least one sensor, and the central processor; and
a connector;
printed circuitry connecting the plurality of electrodes and the electronic processor; and
at least one remote computing processor with an embedded computing programing product operatively connected to the electronic processor;
wherein the at least one battery comprises graphene material;
wherein the plurality of electrodes and electronic processor are imprinted onto the subject's scalp; and
wherein the electronic processor is configured to communicate with the at least one remote computing processor wirelessly;
connecting the at least one remote computing device with the electronic processor using the embedded computing programming product;
collecting brain signals from the subject; and
recording the collected signals on the remote computing device as data.

16. The method of claim 15, further comprising the step of transmitting the data to another remote computing device and making the data available to authorized users.

17. The method of claim 16, wherein the authorized users are doctors, nurses, or researchers.

18. The method of claim 17, further comprising the step of analyzing the data to provide at least one output.

19. A method to produce a brain signal measurement device, comprising:
providing a plurality of electrodes comprising graphene and an epoxy material;
providing an electronic processer, the electronic processor comprises:
at least one printed antenna configured to wirelessly communicate and transmit signals with outside electronic devices;
at least one printed battery configured to provide energy for operation of the electronic processor;
at least one sensor;
a central processor operatively connected to the at least one sensor and the at least one printed antenna to receive signals, process, and transmit received signals;
printed circuitry connecting the at least one printed antenna, the at least one printed battery, the at least one sensor, and the central processor; and
a connector;
providing at least one remote computing processor with an embedded computing programing product operatively connected to the electronic processor;
determining locations for electrodes on a subject's head;
using the three-dimensional printer to print the plurality of electrodes onto the subject scalp's skin at the locations;
using the three-dimensional printer to print the electronic processor onto the subject's scalp skin; and
using the three-dimensional printer to print circuitry to connect the plurality of electrodes with the electronic processor.

20. The method of claim 19, wherein the plurality of electrodes are implanted into the subject scalp's skin at the locations.
